(19) United States
(12) Patent Application Publication
(10) Pub. No.: US 2008/0089936 A1
(43) Pub. Date: Apr. 17, 2008

(54) PROLONGED RELEASE FORMULATION OF ACTIVE PRINCIPLES HAVING A PH-DEPENDENT SOLUBILITY

(75) Inventors: Gerard ALAUX, Beynes (FR); Frederic ANDRE, Verrieres-Le-Buisson (FR); Gareth LEWIS, Dourdan (FR); Veronique SERRE, Igny (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 11/949,291

(22) Filed: Dec. 3, 2007

Related U.S. Application Data
(63) Continuation of application No. PCT/FR2006/001466, filed on Jun. 26, 2006.

(30) Foreign Application Priority Data
Jun. 28, 2005 (FR) 0506539

(51) Int. Cl. A61K 9/22; A61K 31/506; A61K 31/454

(52) U.S. Cl. 424/468; 514/252.14; 514/320

(57) ABSTRACT

The invention concerns a novel formulation for prolonged release of an active principle having a pH-dependent solubility. The inventive formulation comprises a matrix support based on hydrophilic polymer containing a specific dose of active principle, and further comprises one or more acidifying agents in the form of an acid salt of an organic acid.

This application is a continuation of International application No. PCT/FR2006/001,466, filed Jun. 26, 2006, which is incorporated herein by reference in its entirety; which claims the benefit of priority of French Patent Application No. 05/06,539, filed Jun. 28, 2005.

The present invention relates to a novel prolonged-release formulation which can be used for various active ingredients of medicaments.

The invention relates in particular to a novel formulation suitable for the prolonged release of active ingredients which exhibit a pH-dependent solubility and which are used in the form of a base or of addition salts of these bases. In particular, the invention relates to a prolonged-release formulation which can be used with zolpidem, but is not limited to this active ingredient alone.

### BRIEF DESCRIPTION OF THE DRAWINGS

FIG. 1 represents the evolution of the dissolution profiles as a function of time, obtained with the formulation prepared according to Example 3 (12.5 mg zolpidem tablet containing tartaric acid as acidifying agent).

FIG. 2 represents the evolution of the dissolution profiles as a function of time, obtained with the formulation prepared according to Example 2 (12.5 mg zolpidem tablet containing monopotassium tartrate as acidifying agent).

Zolpidem is a short-acting hypnotic agent suitable for the use of the formulation according to the invention. Zolpidem is an active ingredient derived from the chemical family of imidazopyridines. It is administered orally in the form of a tablet.

Zolpidem acts rapidly, with a duration of action ranging up to 6 hours. The pharmacodynamic and pharmacokinetic data show that zolpidem has both a rapid absorption and a rapid bioavailability. Specifically, 70% of the zolpidem is available after oral administration at the therapeutic dose used, which ranges between 5 and 10 mg in conventional forms. The maximum plasma concentration is reached between 0.5 and 3 hours and the half-life time is short, with an average of 2.4 hours.

Immediate-release administration forms of zolpidem are already known. They allow the formulation to disintegrate rapidly in the gastrointestinal tract, to dissolve in the fluids of the tract, allowing the active ingredient to then be absorbed, and to produce its pharmacological effect by inducing sleep in the patient.

A prolonged-release administration form of zolpidem, which makes it possible to release the active ingredient over a period compatible both with the desired sleep period and that required for the elimination of the medicament from the human body, is also known.

Such a prolonged-release form is in particular described in document EP-A-1 135 125, which describes a prolonged-release tablet comprising a single layer containing the active ingredient embedded in the mass of a cellulose-based polymeric material. This polymeric material, which, on contact with aqueous media, forms a matrix, makes it possible to slow down the dissolution of the active ingredient, which can thus produce its pharmacological effect more slowly.

Document EP-A-1 135 125 also describes other embodiments of prolonged-release formulations of zolpidem. For example, a multilayer tablet in which the zolpidem is present in two entities, an immediate-release entity, which is for example external, and a prolonged-release entity, which is for example internal, is described.

The prolonged-release entity comprises a layer or core of polymeric material, in particular a cellulose-based polymer, which releases the amount of zolpidem that it contains more slowly than the immediate-release entity. According to this embodiment, the total of the two zolpidem doses contained in each entity corresponds to the dose that it is desired to administer to the patient.

In order to ensure a micro-pH sufficiently low to maintain the solubility of the zolpidem independent of the pH of the digestive media encountered, document EP-A-1 135 125 describes the use of an acidifying agent, for example of citric acid, tartaric acid or fumaric acid. In the present patent application, the term “local micro-pH” (or “local pH”) is intended to mean the pH that exists in the immediate environment of the formulation, as it dissolves in the gastrointestinal tract; in particular, in a non-disintegrated portion of the formulation, but in which water has already penetrated at a given time.

These acidifying agents have the drawback, however, of being able to react, under certain conditions, with the cellulose-based polymer(s) present in the formulation.

It has thus been noted, when the formulation is exposed to heat and to moisture, that the acidifying agent can cause partial hydrolysis of the cellulose-based polymer constituting the matricial excipient of the formulation, resulting in shorter polymeric chains. This degradation of the polymer by the acidifying agent can then result in reduced stability of the formulations, and can force the manufacturer to recommend stricter storage conditions, with respect to heat and moisture, or to provide for a packaging system that is more airtight/watertight.

The aim of the invention is to solve this drawback, by providing a prolonged-release formulation that makes it possible to maintain a local pH sufficiently low to ensure a solubility of the active ingredient independent of the pH, and without degradation of the polymer present in the matrix, or any negative effect on the stability of the active ingredient.

A first subject of the invention relates to such a prolonged-release formulation.

The formulation of the invention for prolonged-release of an active ingredient having a pH-dependent solubility comprises a matricial excipient based on a hydrophilic polymer which contains a given dose of active ingredient, and is characterized in that it comprises one or more acidifying agents in the form of an acid salt of an organic acid. The acid salt of the acidifying agent can, for example, be an acid salt of citric acid, tartaric acid, fumaric acid, succinic acid or malic acid, and mixtures thereof.

It has in fact been discovered that such acidifying agents, while making it possible to maintain a low local pH, and thus to ensure release of the active ingredient independent of the pH, do not cause any degradation of the hydrophilic polymeric matrix with which they are in contact.

Examples of an acidifying agent in the form of an acid salt are monopotassium tartrate (or potassium bitartrate), monosodium tartrate, monosodium citrate, bisodium citrate, and/or mixtures thereof. Among the acid salts above, monopotassium tartrate also has the advantage of not having any effect on the stability of the zolpidem tartrate or hemitartrate.

The percentage of acidifying agent is between approximately 2% and approximately 10% by weight, calculated relative to the total weight of the formulation, for example between approximately 4% and approximately 8% by weight.

It goes without saying that the invention is not limited to the formulation of zolpidem. Other active ingredients whose solubility depends on the surrounding pH can be used in the context of the invention. This is the case in particular of basic active ingredients present in the formulation in the form of a free base or of a salt, these active ingredients being insoluble or weakly soluble in aqueous media at neutral pH, and when the pKa value is greater than 2.

For example, other active ingredients that can be used with the invention are N-[2-[(4-aminocarbonyl)pyrimidin-2-yl]amino]ethyl]-2-[[3-[4-(5-chloro-2-methoxyphenyl)piperazin-1-yl]propyl]amino]pyrimidine-4-carboximide, 5-(8-amino-7-chloro-2,3-dihydro-1,4-benzodioxin-5-yl)-3-[1-(2-phenylethyl)piperidin-4-yl]-1,3,4-oxodiazole-2(3H)-one hydrochloride, 7-fluoro-2-oxo-4-[2-[4-(thieno[3,2-c]pyridin-4-yl)piperazin-1-yl]ethyl]-1,2-dihydroquinoline-1-acetamide, clopidogrel and mizolastine.

The active ingredients that can be used in the context of the invention can exist in the form of a base, of an addition salt, in particular an acid salt, of a hydrate or of a solvate, i.e., in the form of associations or of combinations with one or more molecules of water or with a solvent. For example, the zolpidem can be used in hemitartrate form.

The formulation of the invention comprises a matricial excipient allowing prolonged release of the active ingredient. By way of examples of a matricial excipient based on a hydrophilic polymer, mention may be made of cellulose and derivatives thereof, for example, hydroxypropylmethylcellulose (hypromellose), hydroxyethylcellulose, hydroxypropylcellulose or carboxymethylcellulose, plant gums and derivatives thereof, alginic acid derivatives, and starch and derivatives thereof.

The prolonged-release formulation of the invention also comprises the conventional ingredients used in this type of formulation. Thus, the formulation of the invention can comprise one or more diluents, disaggregating agents, binders, lubricants, flow excipients, and coloring agents chosen from those known to those skilled in the art.

By way of examples of diluents, mention may be made of lactose and derivatives thereof, for example lactose monohydrate, cellulose and derivatives thereof, for example, microcrystalline cellulose, mannitol, calcium hydrogen phosphate, tricalcium phosphate, calcium sulfate, and starch and derivatives thereof, in particular pregelatized starch and crosslinked starch.

By way of examples of binders, mention may be made of low molecular weight hydroxypropylmethylcellulose and derivatives thereof, for example, Methocel® E5, and povidones, for example, Kollidon K30.

By way of examples of disaggregating agents, mention may be made of starch and derivatives thereof, for example, sodium carboxymethyl starch, crosslinked povidone, croscarmellose, and low molecular weight hydroxypropylcellulose.

By way of examples of lubricants, mention may be made of stearic acid and salts thereof, for example, magnesium stearate, sodium stearyl fumarate, and glyceryl behenate.

By way of examples of flow excipients, mention may be made of silica and derivatives thereof, for example, anhydrous colloidal silica, colloidal silicon dioxide, and talc.

By way of examples of coloring agents, mention may be made of metal oxides, for example, iron, in particular red or yellow iron oxides, indigotine, curcumin, riflavine, tartrazine, azorubine, carmines, erythrosine, red 2G, patent blue V, chlorophylls, copper complexes of chlorophyll, carotenoids, extracts of paprika, anthocyans, titanium dioxide, aluminum, silver, gold, and other pharmaceutically acceptable coloring agents.

According to one embodiment, the formulation of the invention comprises a film-coating layer. This film-coating layer can comprise one or more opacifiers, plasticizers, dyes, and also one or more film-forming polymer excipients.

These ingredients are chosen from those known to those skilled in the art. By way of examples of opacifiers, mention may be made of titanium oxide; by way of examples of plasticizers, mention may be made of polyethylene glycol and derivatives thereof; by way of examples of excipients, mention may be made of lactose and derivatives thereof, for example, lactose monohydrate. By way of examples of film-forming polymer excipients, mention may be made of hydroxypropylethylcellulose and polyvinyl alcohol.

In general, the formulation of the invention comprises a prolonged-release entity and, optionally, an immediate-release entity.

According to the invention, the term “immediate-release entity” is intended to mean a dose comprising a given amount of an immediate-release active ingredient, such as, for example, an immediate-release tablet or pellet, or several of these doses formulated in a gelatine capsule or a tablet; an immediate-release matrix in a tablet; an immediate-release layer in a multilayer tablet; an immediate-release coating layer in a coated tablet.

The term “prolonged-release entity” is intended to mean a dose comprising a given dose of prolonged-release active ingredient, such as a prolonged-release tablet, or several of these doses formulated in a gelatine capsule or a tablet; a prolonged-release matrix in a tablet; a prolonged-release layer in a multilayer tablet.

The unit administration forms of the formulation of the invention comprise oral forms such as tablets, in particular multilayer, coated tablets with a core; soft or hard gelatine capsules.

According to one embodiment, the formulation of the invention consists of a multilayer tablet, in particular a two-layer tablet.

The first layer is an immediate-release layer and comprises one dose of zolpidem, which delivers, by rapid disintegration upon contact with water, all the zolpidem that it contains. The second layer is a prolonged-release layer and also contains one dose of zolpidem, which gradually delivers the active ingredient by erosion and diffusion. The contents of active ingredient of each of the two layers can vary; in general, the respective doses are substantially of the same order of magnitude.

For example, the immediate-release layer can contain from 40% to 55% of the total dose of active ingredient contained in the formulation, for example 48% of active ingredient, and the prolonged-release layer can contain from 45% to 60% of the total dose of active ingredient contained in the formulation, for example 52%.

Thus, according to one embodiment, the formulation of the invention comprises:

a first immediate-release layer comprising an active ingredient whose solubility is pH-dependent, and one or more binders, the first layer optionally containing one or more other excipients such as diluents, disaggregating agents, lubricants or coloring agents;

a second prolonged-release layer, adjacent to the first layer, comprising an active ingredient whose solubility is pH-dependent, one or more acidifying agents in the form of an acid salt, and one or more matricial excipients, the second layer optionally containing one or more other excipients such as diluents, binders, lubricants or coloring agents.

According to one embodiment, the first immediate-release layer comprises, as percentage by weight relative to the total weight of the layer under consideration:

from 1% to 10% by weight of zolpidem hemitartrate,

from 50% to 95% by weight of diluent,

from 0% to 10% by weight of disaggregating agent,

from 0% to 5% by weight of binder,

from 0.5% to 2.5% by weight of lubricant,

from 0% to 0.5% by weight of flow agent,

from 0% to 1% by weight of coloring agent.

According to one embodiment, the second prolonged-release layer comprises, as percentage by weight relative to the total weight of the layer under consideration:

from 1% to 10% by weight of zolpidem hemitartrate,

from 40% to 80% by weight of diluent,

from 20% to 50% by weight of matricial excipient,

from 5% to 15% by weight of acidifying agent,

from 0.5% to 2.5% by weight of lubricant,

from 0% to 0.5% by weight of flow agent.

According to this embodiment, the formulation can also comprise a film-coating layer, coating the first and the second layers. This film-coating layer can be made from commercially available compositions, for example the products sold under the names Opadry® II 32F20797 and Opadry® YS-1-1418, and generally comprises, as main ingredients, a filler excipient, a polymeric binder, an opacifier, a plasticizer, a coloring agent and a solvent.

The excipients contained in the first and the second layer may be identical to or different from one another, with the exception of the acidifying agent.

The compositions according to the invention can be prepared according to methods known to those skilled in the art. The immediate-release tablets can be prepared by direct compression of mixtures of the active ingredients in the form of a base or of salts with diluents, such as microcrystalline cellulose, mannitol, sorbitol or lactose. Other excipients, such as disaggregating agents or lubricants, can be added. The choice between the functional excipients and also the diluents is well known to those skilled in the art.

According to another embodiment, the tablets can be prepared by water-granulation of a mixture of the active ingredient(s) with the appropriate diluents, disaggregating agents and binding polymer, then calibration and drying of the granulated material obtained, and addition of a lubricant, followed by compression on a tablet-compressing machine.

The methods used are generally those described in the literature, for example B. B. Sheth, F. J. Bandelin, R. J F. Shangraw, Compressed tablets, in Pharmaceutical dosage forms: Tablets, Vol 1, edited by H. A. Lieberman and L. Lachman, Dekker N, Y. (1980).

The prolonged-release tablets can be prepared by incorporation of matricial excipients into the formulation, without disaggregating agents. Such matricial excipients are, for example, the hydrophilic polymers described above, for example, based on cellulose, which swell upon contact with water and release the active ingredient in a prolonged manner, by diffusion through the swollen polymer network.

The methods for preparing tablets with several layers or with several coatings are described by W. C. Gunsel, compression coated and layer tablets in pharmaceutical dosage forms: Tablets, Vol 1, edited by H. A. Lieberman and L. Lachman, Dekker N.Y. (1980).

### EXAMPLE 1

### Preparation of a Two-Layered Tablet Containing a 6.25 Mg Dose of Zolpidem

A tablet having the composition indicated in Table I below is prepared according to the techniques described above.

TABLE I

| Constituents | Weight (percentage by layer) |
|---|---|
| Immediate-release layer | |
| Zolpidem tartrate | 2.4 |
| Lactose monohydrate | 70.05 |
| Microcrystalline cellulose | 20.0 |
| Sodium carboxymethyl starch | 3.8 |
| Hypromellose 6 m · Pa · s | 2.5 |
| Magnesium stearate | 1.0 |
| Anhydrous colloidal silica | 0.2 |
| Iron oxide | 0.049 |
| Purified water | q.s. |
| Subtotal | 100.00 |
| Prolonged-release layer | |
| Zolpidem tartrate | 2.6 |
| Lactose monohydrate | 38.2 |
| Hypromellose 4000 m · Pa · s | 30.0 |
| Microcrystalline cellulose | 20.0 |
| Monopotassium tartrate | 8.0 |
| Magnesium stearate | 1.0 |
| Anhydrous colloidal silica | 0.2 |
| Purified water | q.s. |
| Subtotal | 100.00 |
| Film-coating | |
| Lactose monohydrate | 36.0 |
| Hypromellose 15 m · Pa · s | 28.0 |
| Titanium oxide | 24.63 |
| Polyethylene glycol 3350 | 10.0 |
| Iron oxide | 1.37 |
| Purified water | q.s. |
| Subtotal | 100.00 |
| Total mass of the tablet (mg) | 260.00 |

### EXAMPLE 2

### Preparation of a Two-Layered Tablet Containing a 12.5 Mg Dose of Zolpidem

A tablet having the composition indicated in Table II below is prepared according to the techniques described above.

TABLE II

| Constituents | Weight (percentage by layer) |
|---|---|
| Immediate-release layer | |
| Zolpidem tartrate | 4.8 |
| Lactose monohydrate | 67.65 |
| Microcrystalline cellulose | 20.0 |
| Sodium carboxymethyl starch | 3.8 |
| Hypromellose 6 m · Pa · s | 2.5 |
| Magnesium stearate | 1.0 |
| Anhydrous colloidal silica | 0.2 |
| Iron oxide | 0.049 |
| Purified water | q.s. |
| Subtotal | 100.00 |
| Prolonged-release layer | |
| Zolpidem tartrate | 5.2 |
| Lactose monohydrate | 40.6 |
| Hypromellose 4000 m · Pa · s | 25.0 |
| Microcrystalline cellulose | 20.0 |
| Monopotassium tartrate | 8.0 |
| Magnesium stearate | 1.0 |
| Anhydrous colloidal silica | 0.2 |
| Purified water | q.s. |
| Subtotal | 100.00 |
| Film-coating | |
| Lactose monohydrate | 36.0 |
| Hypromellose 15 m · Pa · s | 28.0 |
| Titanium oxide | 20.54 |
| Polyethylene glycol 3350 | 10.0 |
| Indigotine | 5.46 |
| Purified water | q.s. |
| Subtotal | 100.00 |
| Total mass of the tablet (mg) | 260.00 |

### EXAMPLE 3

### Preparation of a Two-Layered Tablet Containing 12.5 Mg of Zolpidem Tartrate and Tartaric Acid

A tablet having the composition indicated in Table III below is prepared according to the techniques described above.

TABLE III

| Constituents | Weight (percentage by layer) |
|---|---|
| Immediate-release layer | |
| Zolpidem tartrate | 4.8 |
| Lactose monohydrate | 67.65 |
| Microcrystalline cellulose | 20.0 |
| Sodium carboxymethyl starch | 3.8 |
| Hypromellose 6 m · Pa · s | 2.5 |
| Magnesium stearate | 1.0 |
| Anhydrous colloidal silica | 0.2 |
| Iron oxide | 0.049 |
| Purified water | q.s. |
| Subtotal | 100.00 |
| Prolonged-release layer | |
| Zolpidem tartrate | 5.2 |
| Lactose monohydrate | 40.2 |
| Hypromellose 4000 m · Pa · s | 25.0 |
| Microcrystalline cellulose | 20.0 |
| Tartaric acid | 8.4 |
| Magnesium stearate | 1.0 |
| Anhydrous colloidal silica | 0.2 |
| Purified water | q.s. |
| Subtotal | 100.00 |
| Film-coating | |
| Hypromellose 15 m · Pa · s | 68.0 |
| Titanium oxide | 21.6 |
| Polyethylene glycol 3350 | 8.0 |
| Iron oxide | 1.4 |
| Polysorbate 80 | 1.0 |
| Purified water | q.s. |
| Subtotal | 100.00 |
| Total mass of the tablet (mg) | 260.00 |

### EXAMPLE 4

### Study of Stability of the Formulations of the Invention

The stability of the formulations according to the invention is compared with that of a formulation prepared according to Example 3.

The experimental conditions are as follows:

bucket apparatus

rotational speed: 100 rpm

temperature: 37° C., 0.5° C.

test sample: one tablet to be analyzed per container

dissolving time: 6 hours

sampling: continuous

reading: every 5 minutes up to 30 minutes, then every 15 minutes up to 6 hours

assay: by UV spectrophotometry at 295 nm (cuvette: 10 mm, filter: 0.22 μm)

standard: solution at 25 mg/l of zolpidem tartrate expressed in terms of anhydrous tartrate, working standard, in 0.01 M of HCl.

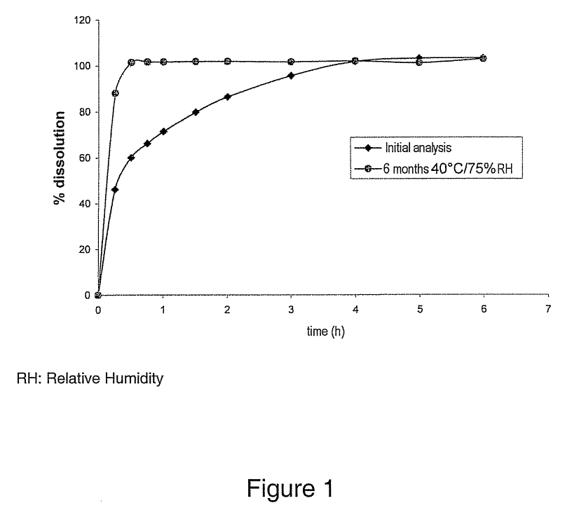

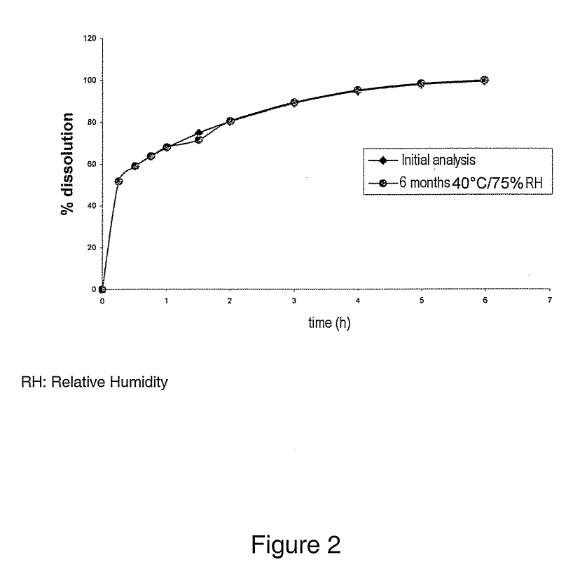

The results obtained are represented in FIG. 1 and FIG. 2, in which FIG. 1 represents the evolution of the dissolution profiles as a function of time, obtained with the formulation prepared according to Example 3 (12.5 mg zolpidem tablet containing tartaric acid as acidifying agent), and FIG. 2 represents the evolution of the dissolution profiles as a function of time, obtained with the formulation prepared according to Example 2 (12.5 mg zolpidem tablet containing monopotassium tartrate as acidifying agent).

It appears that the formulation of Example 2 has a heat and moisture stability greater than that of the formulation prepared according to Example 3.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A prolonged-release formulation that can be used with an active ingredient having a pH-dependent solubility comprising a matricial excipient based on a hydrophilic polymer, wherein the matricial excipient is containing a given dose of active ingredient, and wherein the active ingredient further comprises one or more acidifying agents in the form of an acid salt of an organic acid.

2. The formulation according to claim 1, wherein the acid salt of the acidifying agent is an acid salt of citric acid, tartaric acid, fumaric acid, succinic acid or malic acid, and mixtures thereof.

3. The formulation according to claim 2, wherein the acid salt of the acidifying agent is monopotassium tartrate, monosodium tartrate, monosodium citrate, bisodium citrate, and mixtures thereof.

4. The formulation according to claim 2, wherein the percentage of acidifying agent is from about 2% to about 10% by weight relative to the total weight of the formulation.

5. The formulation according to claim 3, wherein the percentage of acidifying agent is from about 2% to about 10% by weight relative to the total weight of the formulation.

6. The formulation according to claim 2, wherein the percentage of acidifying agent is from about 4% to about 8% by weight relative to the total weight of the formulation.

7. The formulation according to claim 3, wherein the percentage of acidifying agent is from about 4% to about 8% by weight relative to the total weight of the formulation.

8. The formulation according to claim 1, wherein the active ingredient is chosen from zolpidem, N-[2-[(4-aminocarbonyl)pyrimidin-2-yl]amino]ethyl]-2-[[3-[4-(5-chloro-2-methoxyphenyl)piperazin-1-yl]propyl]amino]pyrimidine-4-carboximide, 5-(8-amino-7-chloro-2,3-dihydro-1,4-benzodioxin-5-yl)-3-[1-(2-phenylethyl)piperidin-4-yl]-1,3,4-oxodiazole-2(3H)-one hydrochloride, 7-fluoro-2-oxo-4-[2-[4-(thieno[3,2-c]pyridin-4-yl)piperazin-1-yl]ethyl]-1,2-dihydroquinoline-1-acetamide, clopidogrel and mizolastine.

9. The formulation according to claim 2, wherein the active ingredient is chosen from zolpidem, N-[2-[(4-aminocarbonyl)pyrimidin-2-yl]amino]ethyl]-2-[[3-[4-(5-chloro-2-methoxyphenyl)piperazin-1-yl]propyl]amino]pyrimidine-4-carboximide, 5-(8-amino-7-chloro-2,3-dihydro-1,4-benzodioxin-5-yl)-3-[1-(2-phenylethyl)piperidin-4-yl]-1,3,4-oxodiazole-2(3H)-one hydrochloride, 7-fluoro-2-oxo-4-[2-[4-(thieno[3,2-c]pyridin-4-yl)piperazin-1-yl]ethyl]-1,2-dihydroquinoline-1-acetamide, clopidogrel and mizolastine.

10. The formulation according to claim 3, wherein the active ingredient is chosen from zolpidem, N-[2-[(4-aminocarbonyl)pyrimidin-2-yl]amino]ethyl]-2-[[3-[4-(5-chloro-2-methoxyphenyl)piperazin-1-yl]propyl]amino]pyrimidine-4-carboximide, 5-(8-amino-7-chloro-2,3-dihydro-1,4-benzodioxin-5-yl)-3-[1-(2-phenylethyl)piperidin-4-yl]-1,3,4-oxodiazole-2(3H)-one hydrochloride, 7-fluoro-2-oxo-4-[2-[4-(thieno[3,2-c]pyridin-4-yl)piperazin-1-yl]ethyl]-1,2-dihydroquinoline-1-acetamide, clopidogrel and mizolastine.

11. The formulation according to claim 1, wherein the acid salt of the acidifying agent is monopotassium tartrate, and the active ingredient is zolpidem hemitartrate.

12. The formulation according to claim 1, wherein the polymer forming the matricial excipient is chosen from cellulose and derivatives thereof.

13. The formulation according to claim 12, wherein the cellulose and derivatives thereof is chosen from hydroxypropylmethylcellulose, hydroxyethylcellulose, hydroxypropylcellulose or carboxymethylcellulose, plant gums and derivatives thereof, alginic acid derivatives, starch and derivatives thereof.

14. The formulation according to claim 1, wherein it further comprises one or more diluents, disaggregating agents, binders, lubricants, flow excipients, and coloring agents.

15. A formulation comprising:
a first immediate-release layer comprising an active ingredient whose solubility is pH-dependent, and one or more binders, the first layer optionally containing one or more other excipients such as diluents, disaggregating agents, lubricants or coloring agents; and
a second prolonged-release layer, adjacent to the first layer, comprising an active ingredient whose solubility is pH-dependent, one or more acidifying agents in the form of an acid salt, and one or more matricial excipients, the second layer optionally containing one or more other excipients such as diluents, binders, lubricants or coloring agents.

16. The formulation according to claim 15, wherein the first immediate-release layer comprises, as percentage by weight relative to the total weight of the layer under consideration:
from about 1% to about 10% by weight of zolpidem hemitartrate,
from about 50% to about 95% by weight of diluent,
from about 0% to about 10% by weight of disaggregating agent,
from about 0% to about 5% by weight of binder,
from about 0.5% to about 2.5% by weight of lubricant,
from about 0% to about 0.5% by weight of flow agent, and
from about 0% to about 1% by weight of coloring agent.

17. The formulation according to claim 15, wherein the second prolonged-release layer comprises, as percentage by weight relative to the total weight of the layer under consideration:
from about 1% to about 10% by weight of zolpidem hemitartrate,
from about 40% to about 80% by weight of diluent,
from about 20% to about 50% by weight of matricial excipient,
from about 5% to about 15% by weight of acidifying agent,
from about 0.5% to about 2.5% by weight of lubricant, and
from about 0% to about 0.5% by weight of flow agent.

18. The formulation according to claim 15, wherein it further comprises a film-forming layer comprising, as main ingredients, a filler excipient, a polymeric binder, an opacifier, a plasticizer, a coloring agent and a solvent.

19. A formulation comprising:
a first immediate-release layer comprising, expressed as percentage by weight relative to the total weight of the said layer:
4.8% of zolpidem hemitartrate,
67.65% of lactose monohydrate,
20.0% of microcrystalline cellulose,
3.8% of sodium carboxymethyl starch,
2.5% of hydroxypropylmethylcellulose 6 m.Pa.s,
1.0% of magnesium stearate,
0.2% of anhydrous colloidal silica,
0.049% of iron oxide, and
q.s. purified water,
a second prolonged-release layer comprising, expressed as percentage by weight relative to the weight of the second layer:
5.2% of zolpidem hemitartrate,
40.6% of lactose monohydrate,
25.0% of hydroxypropylmethylcellulose 4000 m.Pa.s,
20.0% of microcrystalline cellulose,
8.0% of monopotassium tartrate,
1.0% of magnesium stearate,
0.2% of anhydrous colloidal silica, and
q.s. purified water, and
a film-coating layer comprising, expressed as percentage by weight relative to the weight of the film-coating layer:
36.0% of lactose monohydrate,
28.0% of hydroxypropylmethylcellulose 15 m.Pa.s,
20.54% of titanium oxide,
10.0% of polyethylene glycol 3350,
5.46% of indigotine, and
q.s. purified water.

20. A formulation comprising:
a first immediate-release layer comprising, expressed as percentage by weight relative to the weight of the first layer:
2.4% of zolpidem hemitartrate,
70.05% of lactose monohydrate,
20.0% of microcrystalline cellulose,
3.8% of sodium carboxymethyl starch,
2.5% of hydroxypropylmethylcellulose 6 m.Pa.s,
1.0% of magnesium stearate,
0.2% of anhydrous colloidal silica,
0.049% of iron oxide, and
q.s. purified water,
a second prolonged-release layer comprising, expressed as percentage by weight relative to the weight of the second layer:
2.6% of zolpidem hemitartrate,
38.2% of lactose monohydrate,
30.0% of hydroxypropylmethylcellulose 4000 m.Pa.s,
20.0% of microcrystalline cellulose,
8.0% of monopotassium tartrate,
1.0% of magnesium stearate,
0.2% of anhydrous colloidal silica, and
q.s. purified water, and
a film-coating layer comprising, expressed as percentage by weight relative to the weight of the film-coating layer:
36.0% of lactose monohydrate,
28.0% of hydroxypropylmethylcellulose 15 m.Pa.s,
24.63% of titanium oxide,
10.0% of polyethylene glycol 3350,
1.37% of iron oxide, and
q.s. purified water.

21. The formulation according to claim 1, wherein it is in the form of a tablet, a coated tablet, a multilayer tablet with a core; a soft or hard gelatine capsule.

22. The formulation according to claim 11, wherein it is in the form of a tablet, a coated tablet, a multilayer tablet with a core; a soft or hard gelatine capsule.

23. The formulation according to claim 15, wherein it is in the form of a tablet, a coated tablet, a multilayer tablet with a core; a soft or hard gelatine capsule.

24. The formulation according to claim 19, wherein it is in the form of a tablet, a coated tablet, a multilayer tablet with a core; a soft or hard gelatine capsule.

25. The formulation according to claim 20, wherein it is in the form of a tablet, a coated tablet, a multilayer tablet with a core; a soft or hard gelatine capsule.
